(19) United States
(12) Patent Application Publication
(10) Pub. No.: US 2011/0021925 A1
(43) Pub. Date: Jan. 27, 2011

(54) Mounted vein contrast enchancer

(76) Inventors: Fred Wood, Medford, NY (US); Ron Goldman, Cold Spring Harbor, NY (US); Vincent Luciano, Shoveham, NY (US)

Correspondence Address: Thomas A. O'Rourke;Bodner & O'Rourke, LLP,, Suite 108, 425 Broadhollow Road, Melville, NY, 11747, US

(21) Appl. No.: 12/804,088

(22) Filed: Jul. 13, 2010

Related U.S. Application Data
(63) Continuation-in-part of application No. 11/807,064, filed on May 25, 2007.
Application No. 11/807,064 is a continuation-in-part of application No. 11/938,343, filed on Nov. 12, 2007.
Application No. 11/938,343 is a continuation-in-part of application No. 11/807,255, filed on May 25, 2007.
Application No. 11/807,255 is a continuation-in-part of application No. 11/807,359, filed on May 25, 2007.
Application No. 11/807,359 is a continuation-in-part of application No. 11/700,729, filed on Jan. 31, 2007.
Application No. 11/700,729 is a continuation-in-part of application No. 11/807,057, filed on May 25, 2007.
Application No. 11/807,057 is a continuation-in-part of application No. 11/823,862, filed on Jun. 28, 2007.
Application No. 11/823,862 is a continuation-in-part of application No. 11/478,322, filed on Jun. 29, 2006.
Application No. 11/478,322 is a continuation-in-part of application No. 11/605,069, filed on Nov. 28, 2006.
Application No. 11/605,069 is a continuation-in-part of application No. 11/605,172, filed on Nov. 28, 2006.
Application No. 11/605,172 is a continuation-in-part of application No. 11/605,076, filed on Nov. 28, 2006.
(60) Provisional application No. 61/270,760, filed on Jul. 13, 2009.

(51) Int. Cl. A61B 6/00

(52) U.S. Cl. 600/476

(57) ABSTRACT

A vein-image-enhancing system transmits two different wavelengths of laser light, being directed by a steering means, to form a raster pattern on a target. Reflected light comprises a contrasted image formed by a portion of the first wavelength being absorbed by veins, and a portion of that first wavelength being reflected from the target. A photodectector sensitive to the first wavelength captures reflected light. The contrasted image is projected onto the target to illuminate vein locations using the second wavelength, which comprises visible light. Projection may operate according to one of three modes: the alternating frame mode, a dual buffer mode, and real time mode. The AFM and DBM modes incorporate analog to digital converters and digital memory for timed projection onto the target. The vein enhancer may be specially attached to different platforms—hospital bed, IV stand, wheelchair, etc., to enable convenient use of the enhancer on a patient.

### CROSS-REFERENCES TO RELATED APPLICATIONS

This application claims priority on U.S. Provisional Application Ser. No. 61/270,760, filed on Jul. 13, 2009, the disclosures of which are incorporated herein by reference.

This application is also a continuation-in-part of U.S. application Ser. No. 11/807,064 filed May 25, 2007, U.S. application Ser. No. 11/938,343 Filed Nov. 14, 2007, U.S. application Ser. No. 11/807,255 filed May 25, 2007, U.S. application Ser. No. 11/807,359 filed May 25, 2007, U.S. application Ser. No. 11/700,729 filed Jan. 31, 2007, U.S. application Ser. No. 11/807,057 filed May 25, 2007, U.S. Serial application Ser. No. 11/823,862 filed Jun. 28, 2007, U.S. application Ser. No. 11/478,322 filed Jun. 29, 2006, U.S. application Ser. No. 11/605,069 filed Nov. 28, 2006, U.S. application Ser. No. 11/605,172 filed Nov. 28, 2006, and U.S. application Ser. No. 11/605,076 filed Nov. 28, 2006.

### FIELD OF THE INVENTION

The present invention is directed to improved systems and apparatus for finding veins. More particularly, the present invention relates to a means for having a vein locating device in close proximity to a patient, where the patient is in a variety of locations or a variety of medical situations.

### BACKGROUND OF THE INVENTION

It is fairly routine in hospitals and nursing home settings for a patient to have to receive an injection or be receiving medications, nutrients, etc. from an IV. In order to administer the medication, to receive an IV, the needle must be inserted into a vein. Many patients do not have their veins readily visible so that a needle can be readily inserted into the vein. As a result, there is a need for devices to assist medical personnel in locating veins. There are a number of devices that can be used to facilitate vein location to permit medical personnel to locate the vein and to provide the patient with an injection or fluids through an IV or for other purposes.

Veins need to be located other medical procedures as well. In blood testing a patient must have a vein located so that the collecting receptacle can tap the vein to draw blood. Similarly, there are certain types of injections where the location of a vein must be determined. Besides the foregoing, there are a number of other medical type situations where seeing a vein location is desirable.

Devices that can assist the medical personnel in this regard include but are not limited to the subject matter of U.S. application Ser. No. 11/807,064 filed May 25, 2007, U.S. application Ser. No. 11/985,343 Filed Nov. 14, 2007, U.S. application Ser. No. 11/807,255 filed May 25, 2007, U.S. application Ser. No. 11/807,359 filed May 25, 2007, U.S. application Ser. No. 11/700,729 filed Jan. 31, 2007, U.S. application Ser. No. 11/807,057 filed May 25, 2007, U.S. Serial application Ser. No. 11/823,862 filed Jun. 28, 2007, U.S. application Ser. No. 11/478,322 filed Jun. 29, 2006, U.S. application Ser. No. 11/605,069 filed Nov. 28, 2006, U.S. application Ser. No. 11/605,172 filed Nov. 28, 2006, and U.S. application Ser. No. 11/605,076 filed Nov. 28, 2006, the disclosures of which are incorporated herein by reference.

### SUMMARY OF THE INVENTION

The present invention provides a platform for mounting a vein enhancing device so that medical personnel can have ready access to the vein enhancing device for use with a patient. There is no need for personnel to carry the device on their rounds where it can be lost or dropped. Time is saved because there is no need to search for the device in a patient's room at a nursing station or elsewhere on the premises.

The platform may be either in a fixed location or a mobile platform that can be used by emergency personnel with patients in a variety of medical situations including but not limited to EMT personnel, corpsmen in the field of battle, on stretchers, vehicle transport devices etc.

The vein enhancing device of the present invention may be mounted on a variety of platforms including but not limited to:

### 1. Hospital bed

2. IV pole mount
3. bed rail
4. holding compartment inside bed rail
5. holding compartment under bed
6. stretcher
7. chair including a wheel chair
8. ICU boom
9. lighting boom
10. medical cart
11. room lighting fixtures
12. iv pole standard mounts

Where the vein enhancing device is connected to a bed, chair, gurney or other device, the vein enhancing device may be positioned in any suitable location including but not limited to:

1. Left side
2. right side
3. head
4. foot
5. dropped into existing IV pole sockets
6. rail attachment
7. embedded in side rail
8. holding compartment in rail

There may also be a foot petal release for gooseneck or support member. There may be control buttons on bed. The controls may also permit the user to dim room lighting to facilitate viewing the device.

The mounting to the platform may be by any suitable means including but not limited to:

1. magnetic attachment
2. key lock

The power to the vein enhancing device may be provided by any suitable means including but not limited to:

1. wiring built into bed
2. Wiring permitting the device to communicate with electronic controls in bed
3. Bed providing power to unit, for charging and or operation
4. bed provides power only to unit, without battery
5. ICU boom, lighting boom or other boom provides power/wiring to the device
6. wiring inside IV pole
7. electrical connections on each pole or mounting point

### BRIEF DESCRIPTION OF THE DRAWINGS

FIG. 1 shows the vein enhancing device of the present invention mounted on a hospital bed.

FIG. 2 shows the vein enhancing device of the present invention mounted to an IV pole.

FIG. 3 shows the vein enhancing device of the present invention attached to a bed rail.

FIG. 4 shows the vein enhancing device of the present invention emanating from a holding component inside a bed rail of a bed frame.

FIG. 5 shows the vein enhancing device of the present invention emanating from a holding compartment under a bed or a bed frame.

FIG. 6 shows a vein enhancing device of the present invention mounted on a stretcher.

FIG. 7 shows the vein enhancing device of the present invention mounted onto a chair.

FIG. 8 shows the vein enhancing device of the present invention secured to an ICU boom.

FIG. 9 shows the vein enhancing device of the present invention mounted to a lighting boom.

FIG. 10 shows the vein enhancing device of the present invention mounted to a medical cart.

FIG. 11 shows an alternative embodiment of the vein enhancing device of the present invention hanging from a boom.

FIG. 12 shows the vein enhancing device of the present invention secure to a platform by means of a locking member.

FIG. 13 shows the vein enhancing device of the present invention, as the sleeve is being inserted into the collar of the platform.

FIG. 14 illustrates one example of the vein scanner that may be used with the present invention.

FIG. 15 shows a control block diagram for the vein scanner of FIG. 14.

FIG. 16 shows the Dual Buffer Mode of operation of the vein scanner of FIG. 14.

FIG. 17 depicts the Real Time Mode of operation of the vein scanner of FIG. 14.

### DETAILED DESCRIPTION OF THE PRESENT INVENTION

The present invention is directed to a platform for supporting a vein enhancing device. In one embodiment the platform may also support a patient. In another embodiment the platform may be in proximity to a patient whereby the platform can bring the vein enhancing device to the patient or the patient to the vein enhancing device. The platform may be mobile or fixed. In addition, the platform may provide power to the vein enhancing device. The power may be supplied by a battery, solar power or the platform may receive power from a fixed or movable source such as a wall socket or plug or a mobile power device.

The vein enhancing device may have a housing having a least one surface that houses the vein enhancing components. These components and their operation are described in U.S. application Ser. No. 11/807,064 filed May 25, 2007, U.S. application Ser. No. 11/935,343 Filed Nov. 14, 2007, U.S. application Ser. No. 11/807,255 filed May 25, 2007, U.S. application Ser. No. 11/807,359 filed May 25, 2007, U.S. application Ser. No. 11/700,729 filed Jan. 31, 2007, U.S. application Ser. No. 11/807,057 filed May 25, 2007, U.S. Serial application Ser. No. 11/823,862 filed Jun. 28, 2007, U.S. application Ser. No. 11/478,322 filed Jun. 29, 2006, U.S. application Ser. No. 11/6058,069 filed Nov. 28, 2006, U.S. application Ser. No. 11/605,172 and U.S. application Ser. No. 11/605,076 filed Nov. 28, 2006. The disclosures of which are incorporated herein by reference.

The housing may have a top surface and a bottom surface and one or more side surfaces. A beam of laser light may be emitted from one of the surfaces to enhance the view of a patient's veins. Extending from the housing may be a support for attaching the housing to a platform. The support may be rigid or flexible as desired. In one embodiment the support may be solid and there may be additional supports secured to the housing to provide electrical power to the laser beam driving means. In another embodiment the support may be hollow and thereby providing a conduit for the necessary electrical wiring to operate the device. The wiring may also be on the outer surface of the support. The support may be secured to the platform by a variety of means. In one embodiment, the support may be a generally tubular member but other configurations are possible as well.

The platform may be any size, or shape, or other configurations. The figures submitted with this application show representative platforms and these figures are not to be deemed limiting of the types of platforms that may be used in connection with the present invention. As indicated above, the platform may be fixed, or mobile or a combination of both such that in one configuration the platform may be mobile and in another configuration the platform maybe in a fixed location. For example in one configuration the platform may be wheeled and have wheels that may be lowered or arranged in a configuration where the wheels are operative to permit the platform to move. Later, the wheels may be raised or rendered inoperative so that the platform may be fixed in a location.

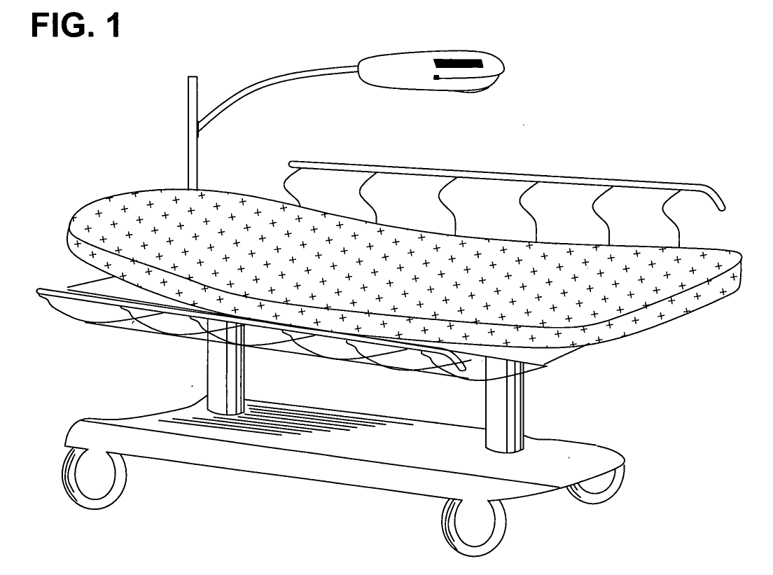

FIG. 1 shows a hospital bed with the vein enhancing device of the present invention secured thereto. The Figure shows the vein enhancing device position in the vicinity of the patients head but it will be appreciated that the support arm of the vein enhancing device may be secured elsewhere on the frame of the hospital bed. The hospital bed has a support for supporting a mattress. Extending from the support is a base that has a plurality of wheels thereon for moving the bed from location to location. The bed may have one or more side rails that may be lowered or raised as desired. The hospital bed may have an IV support for suspending an IV bag therefrom. The IV support may be used to support the vein enhancing device. The vein enhancing device may be supported elsewhere on the bed such as along the top, bottom, or sides of the mattress support, along any of the rails or elsewhere on the bed. The vein enhancer may be battery operated from a battery secured to the frame of the bed or positioned off the frame. Many hospital beds are powered to permit the patient to raise and lower the mattress or parts thereof. The electrical wiring in the bed can provide an electrical connection to the vein enhancing device. The IV pole mount secured to the bed in FIG. 1 may also be free standing and separate from the bed. The free standing IV pole may have a base portion that supports the IV pole on a flat surface. Alternatively the IV pole may be secured to a wall surface or other suitable surface in the area such as countertops, tables, etc.

The vein location device or vein scanner of the present invention may be secured to a platform by a myriad of different means. In one embodiment, the platform may have an orifice in the surface thereof. The vein scanner can be secured to the platform's orifice by a mating member that extends from the scanner. Similarly the platform may have one or more protruding members extending from a surface of the platform. The vein scanner may have a recess that receives the protruding member extending form a surface of the platform. Where there is an orifice and protruding member that is received into the vein scanner the connection may rely on a friction fit or other suitable means to prevent the scanner from being separated from the platform. Other types of connection may include a spring loaded dimple that is pushed inwardly as the extending member is received by the orifice. The spring loaded dimple can provide a friction fit with the inner surface of the orifice or the dimple may be received by a corresponding recess in the orifice to lock it into position. A bayonet type connection can also be used.

The vein scanner can be clamped to the platform or secured directly to the platform. The clamping mechanism may be part of the scanner, part of the platform or a separate clamping means. A variety of clamping means can be used. For example the claiming may include a “C” type clamp, a spring clamp having a first and second arms etc. “C” clamps have a generally C shape and there may be an extending member that extends from one arm of the C to the other arm of the “C” and can be use to secure the “C” clamp to a surface. The extendable member may be threaded or locked into a secure position. Alternatively the arms of the C may be joined by a body member that is expandable so that the arms may be moved from a first position where the arms separated to a second position where the arms are closer together so that a portion of the platform is secured between the two arms of the clamps.

In the spring clamp the arms of the clamp can be joined by a spring. As two ends of the clamp are pushed closer together the opposite ends of the clamp separate to permit a portion of the platform to be inserted between the ends of the clamp that are moving apart. As pressure is released on the first end of the arm of the clamp these two arms separate due to the force of a spring while the ends of the spring that were separated return to their clamping position.

In another type of clamping means there is a strip of suitable material such as metal or plastic. The strip has a first end and a second end and the strip is formed into a generally circular shape having a given diameter. As the clamp is tightened, the diameter of the circular portion is reduced so that the clamp becomes tighter. This type of clamp is particularly useful for securing the vein scanner to a rod or tubular member. As the clamp is tightened the clamp secures the device to the platform.

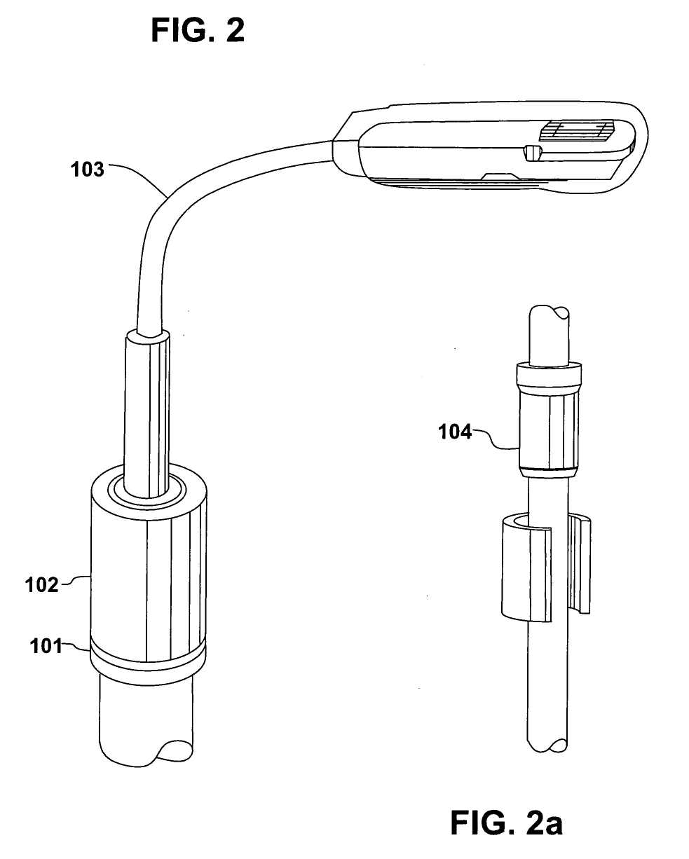

FIG. 2 shows a representative mounting means for securing a support arm of the vein enhancing device to an IV pole mount. In this configuration there is a base with a receiving member 102 extending from the base member 101. The receiving member 102 may receive an end of the support arm which can be secured to the IV pole mount. As seen in FIG. 2 the support arm 103 has a sleeve 104 positioned thereon. The sleeve 104 can be positioned in the receiving member 102. The receiving member may secure the support arm to the IV pole. There are a number of ways, however, that the support arm for the vein enhancing device may be secured to the IV pole.

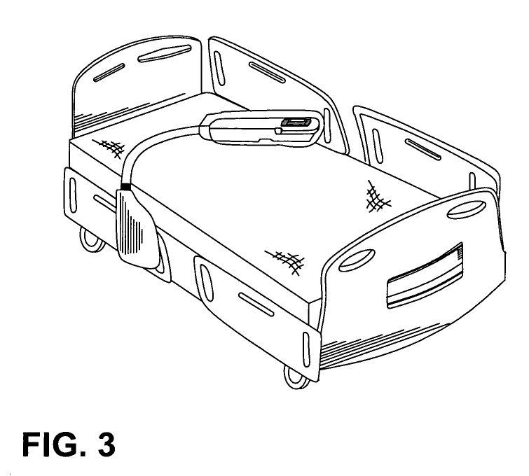

FIG. 3 shows a different style of hospital type bed from the design of FIG. 1. In this embodiment the bed has one or more rails for protecting the patient from falling from the bed. In the embodiment shown in FIG. 3 there is a top or headboard, a footboard, and a pair of side rails. As seen in FIG. 3 the support arm of the vein enhancing device may be secured to one of the side rails. Alternatively it may be secured to the rails or the footboard or headboards by any suitable means. Also, the support arm may be secured to the side rails or the footboard or head board at any position along its upper surface, either of its side surfaces or an end or bottom surface. It will be appreciated that the side rails and/or the foot board may be secured to a frame where the support arm may be secured instead.

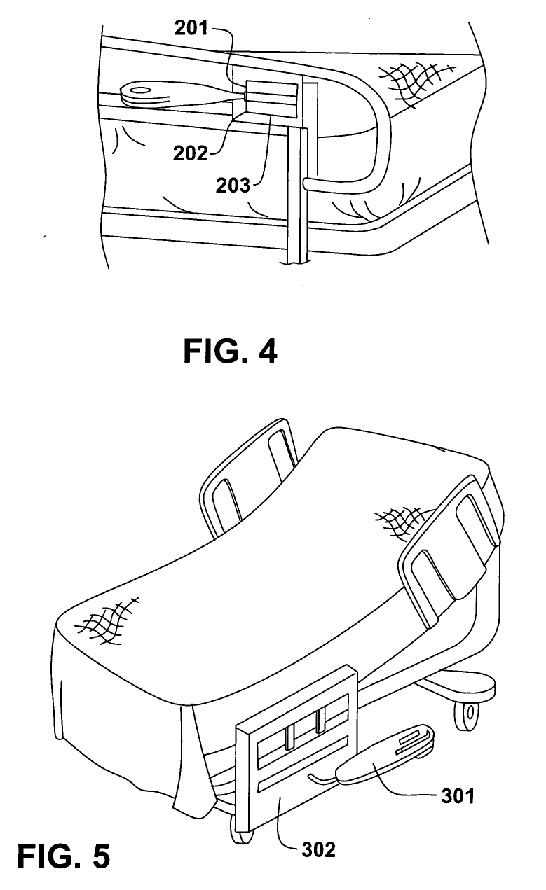

The beds of FIGS. 1 and 3 may be provided with a holding compartment for storage of the vein enhancing device. In FIG. 4 for example there is shown a receptacle for receiving the vein enhancing device. This receptacle may be secured to the bed by any suitable means. As seen in FIG. 4 there is a rear plate 201 that is secured for example to the side rail. The plate has a top end and a bottom end. Extending from the bottom end is generally a horizontal portion 202 that is at an angle, preferably a 90 degree angle to the plate 201. There is a face plate 203 that extends upwardly from the bottom portion 202.

In a preferred embodiment the face plate 203 and the rear plate 201 are generally parallel to each other thereby forming a pocket open at each end for receiving the vein enhancing device. It will be appreciated that there may be numerous other configurations for the holder of the vein enhancing device. In addition, the holder may be positioned in any number of locations both on and off the bed.

An alternative embodiment is shown in FIG. 5 where the vein enhancing device 301 may be stored in a storage compartment 302 that is part of the bed or secured thereto. The compartment may be a drawer or “cubby” that receives a vein enhancing device. The device may be electrically connected to the bed through a suitable means in the compartment or may be removably stored therein. The vein enhancing device is adapted to be secured to an electrical connecting means elsewhere or it may be battery operated. The compartment may have one or more sides and an open area towards an edge of the bed to receive the vein enhancing device. There may be a closed or open rear. In one embodiment the compartment may be a generally cubic structure having a top surface, a bottom surface and a pair of side surfaces. There may be a rear wall enclosing the space formed by the surface. The side opposite the rear wall may open or it may have a door. In still a further embodiment there may be a pair of runners or the inside surfaces that receives a drawer which can receive the vein enhancing device for storage.

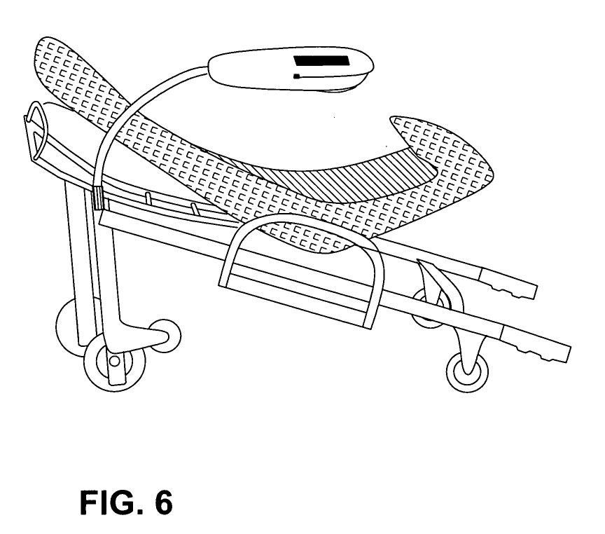

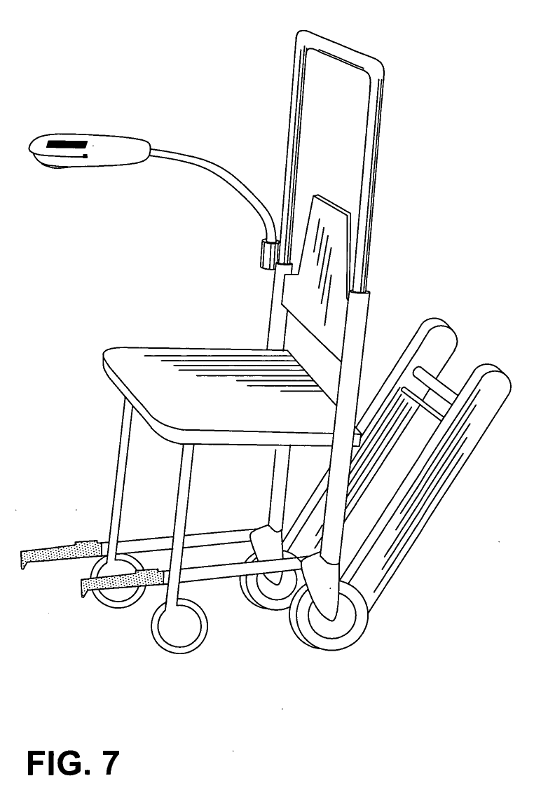

FIG. 6 shows the vein enhancing device mounted to a stretcher. Stretchers, like hospital beds, can have a variety of shapes and sizes and can range from a simple body support surface with one or more holding means to carry the stretcher. The means can be poles, handles, holes, etc. The vein enhancing device may be secured to the stretcher by any suitable means. More elaborate stretchers such as shown in FIG. 6 may also be provided with a vein enhancing device. As seen in FIG. 6 there is a frame that supports a mattress or other body support. The frame may have a variety of configurations. The vein enhancing device may be secured to the frame.

In an alternative embodiment of the present invention the vein enhancing device may be mounted on a wheelchair. The wheel chair may have a patient sitting area with a seat area and preferably a back support. There are a plurality of wheels rotatably mounted on the frame so that the wheel chair is mobile. The vein enhancing device may be secured to the frame of the wheel chair by any suitable means. The wheelchair may have a battery pack secured to the frame to provide electrical power to the vein enhancing device. As seen in the embodiment of FIG. 7, the vein enhancing device may for example have its support arm extending from the back support of the frame. It will be appreciated that the vein enhancing device may be secured elsewhere on the wheel chair or the frame thereof.

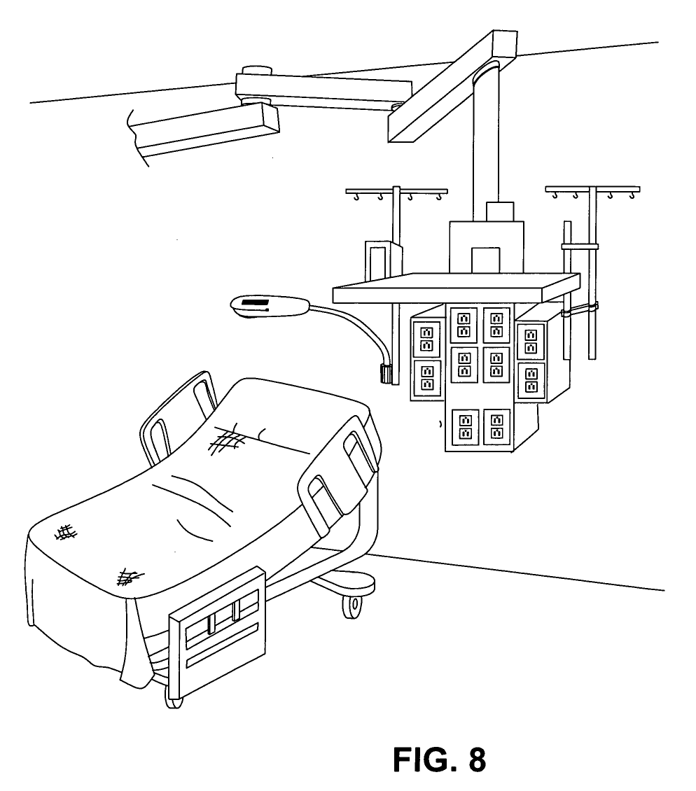

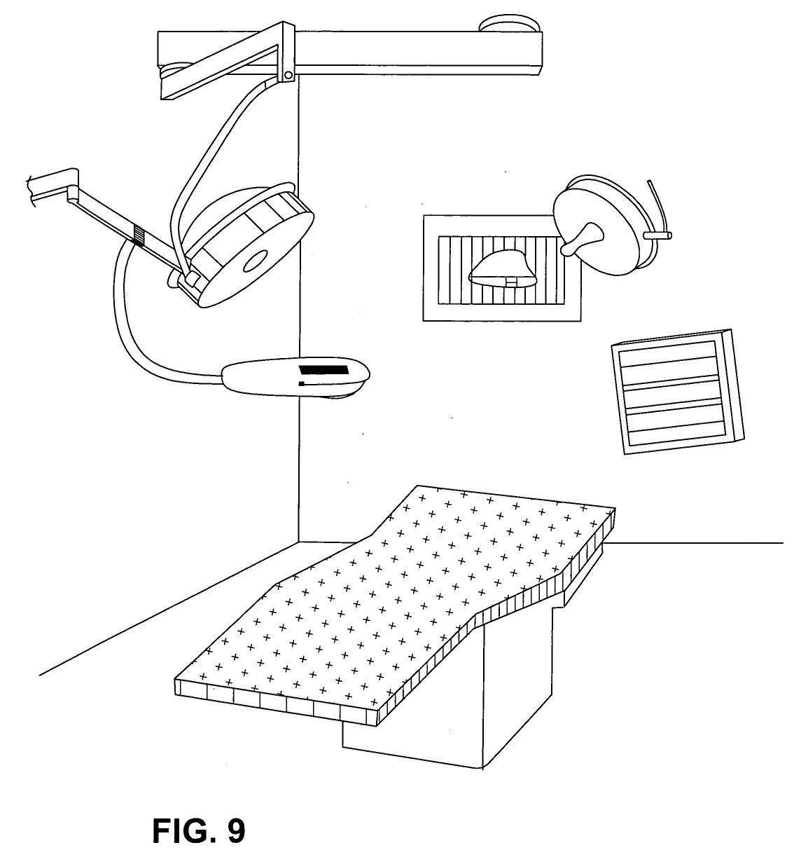

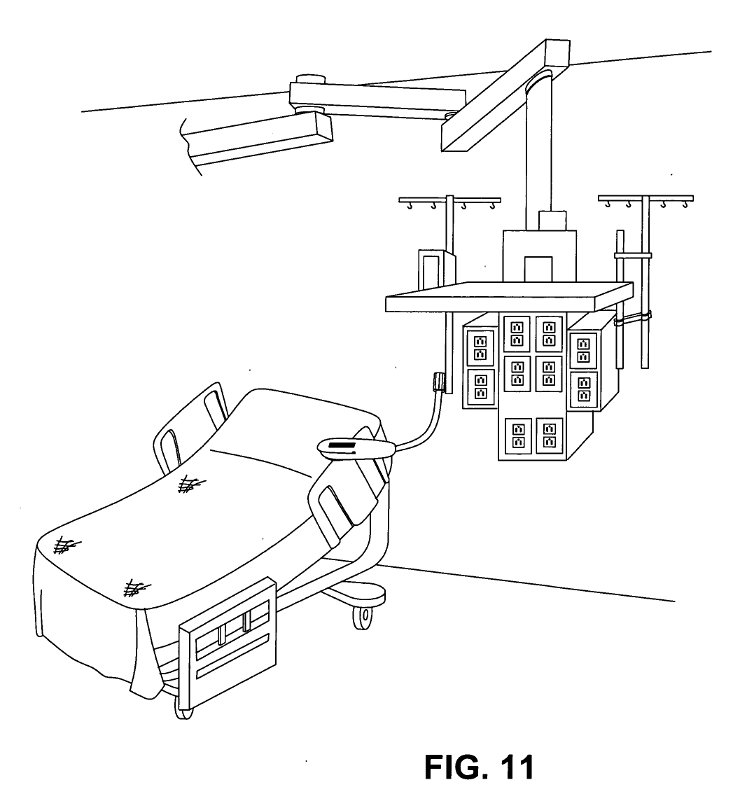

In many patient care settings where critical care is provided there may be a boom or support center that provides electrical power, support arms, oxygen or other gases, etc. An example of such booms is shown in FIGS. 8, 9 and 11. FIG. 8 shows a representative sample of an ICU type boom that has a plurality of electrical and other connecting sockets to hook equipment up thereto. The boom also has one or more support arms for supporting IV bags and other equipment. The boom may be for a single bed or a plurality of beds. The vein enhancing device may be mounted to the boom in a variety of ways. One example is shown in FIG. 8 where the support arm is mounted to an arm extending downwardly from the boom. The boom may have a plurality of orifices for receiving one or more support arms. The orifices receive the support arm and can be tightened around the arm. Alternatively, the support arms may be threadably secured to the orifices. In one of the orifices, the support arm of the vein enhancing device may be positioned. As seen in FIG. 8 the support arm may extend upwardly from the boom. As seen in FIG. 11 the support arm of the vein enhancing device may extend downwardly from the boom. It will be appreciated by those skilled in the art that the vein enhancing device may be secured in any area of the boom as may be convenient. The support arm of the vein enhancing device may also be positioned upwardly or downwardly as desired. The vein enhancing device may be permanently secured to the boom or other platform described herein or it may be removably connected. Besides an ICU type boom there are other types of booms often found in patient care settings. One example of these other type of booms is shown in FIG. 9. FIG. 9 shows a lighting boom. The lighting boom may include an arm that is movably secured to a wall or ceiling surface. The end of the arm opposite the mounting surface may have one or more lighting means extending therefrom. The vein enhancing device of the present invention may be mounted to the arm or the light fixture of the lighting boom. The lighting boom may provide other functions in addition to lighting. The vein enhancing device may be permanently secured to the lightening boom or it may be removably mounted thereto.

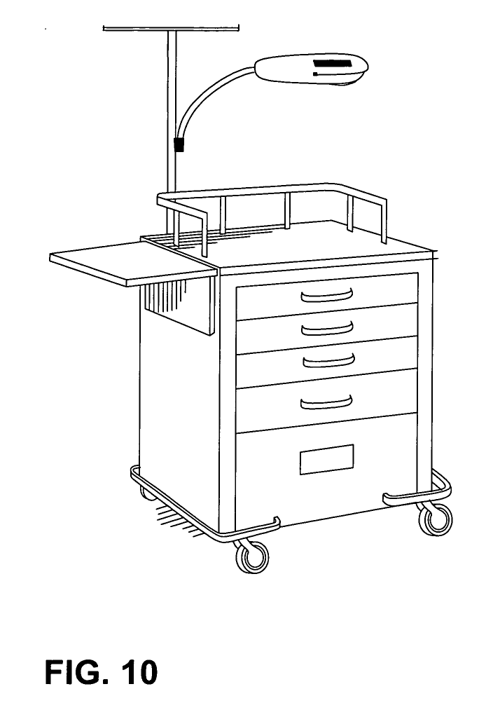

Another example of a platform for supporting a vein enhancing device is a tray table or cart that is used in health related facilities. The cart or table may have a top surface and one or more side surfaces extending downwardly from the top surface. Instead of a side surface there may be legs extending from the top surface. The platform may also have wheels if desired. The vein enhancing device may be mounted to the top platform or to a side thereof by any suitable means. One example of this type of platform is shown in FIG. 10 where there is a top or work surface, a plurality of drawers or doors for storage, the cart may have a plurality of wheels to permit the platform to be readily moved. The vein enhancing device may extend from a top or side surface of the cart. The cart may also have an extension thereon to increase the work space.

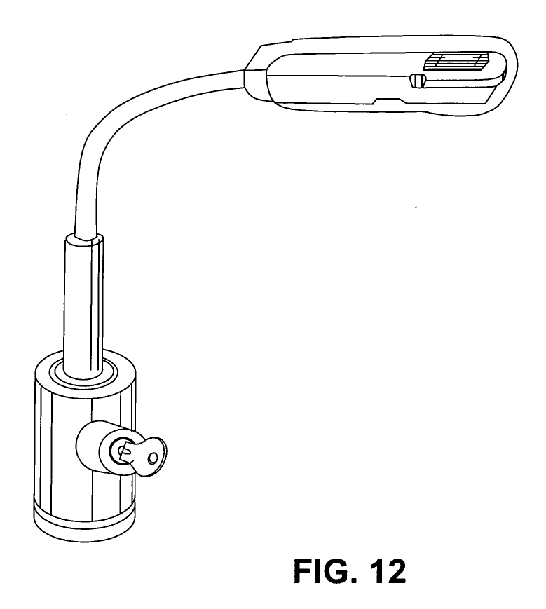

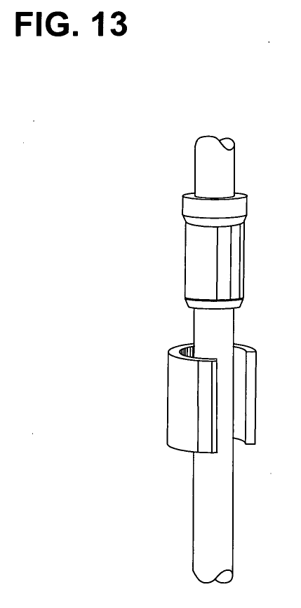

FIG. 12 shows a representative locking system that may be used with support members of the vein enhancing device. There may also be a layered member that secures the support members to a recess area in a cylindrical member. There may be other locking arrangements as well. The locking arrangement prevents theft. FIG. 13 shows an alternative means of securing the support member to a platform.

In other embodiments, the enhancing device of the present invention may have the wiring for the device built into a bed. The device may also communicate with electronic controls on the bed. There may also be a power unit on the bed or bed frame for charging or operating the vein enhancing device. The hospital bed circuitry may also provide power to the device. The device of the present invention may also be interconnected to the bed monitor or other recording device and can record when the device was used, by whom and for the time period. There may also reminders issued by the platform or the device. These reminders can relate to a variety of data including but not limited to reminders for cleaning, testing, etc.

Examples of vein scanners that can be used with the present invention include but are not limited to those that are shown in copending application Ser. No. 11/823,862 filed Jun. 28, 2007 and entitled “Three Dimensional Imaging of Veins, Ser. No. 11/807,057 filed May 25, 2007 entitled Scanned Laser Vein Contrast Enhancer, Ser. No. 11/807,359 filed May 25, 2010 entitled Laser Vein Contrast Enhancer, Ser. No. 11/985,343 filed Nov. 14, 2007 entitled Micro Vein Enhancer and Ser. No. 11/478,322 filed Jun. 29, 2006 entitled Micro Vein Enhancer, 61/271,587 filed Jul. 22, 2010 entitled Vein Scanner, 61/278,948 filed Oct. 14, 2009, entitled Multispectral Detection and Presentation of an Objects Characteristics and 61/279,980 filed Oct. 28, 2009 entitled Modular Mounting Mirror Endoscopy the disclosures of which are incorporated herein by reference.

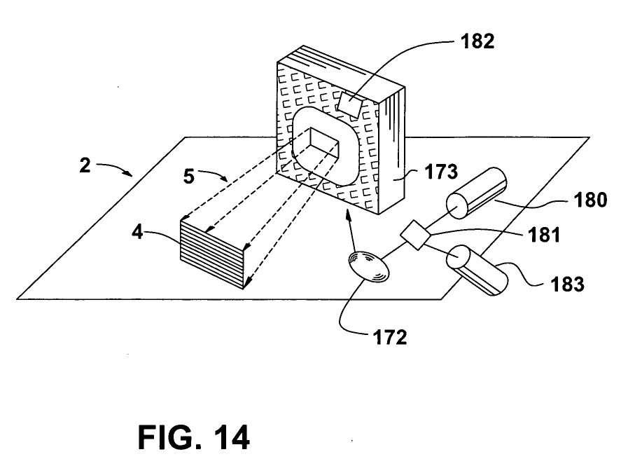

FIG. 14 illustrates an example of one embodiment of the vein scanner that may be used in accordance with the present invention. A single colored laser 180, for example a 630 nm semiconductor red laser, is projected into combiner 181. A semiconductor laser 183 is also projected into the combiner 181. Laser 183 may have a frequency from 700 nm to 1000 nm, with a preferred frequency of 740 nm. An illustrative example of a semiconductor 740 nm laser is Sacher Lasertechnik's Fabry Perot Diode Laser 740 nm, 10 mw, model number FP-0740-10. The combiner 181 outputs a combined laser beam 184 which is the combination of the 630 nm red and the 740 nm laser beams. Combiners for combining two lasers of different frequencies are well known in the art and will not be further described herein. The combined laser beam 184 is positioned to hit off mirror 172 and then to hit the MEMS scanner 173. The MEMS scanner moves in a raster pattern thereby causing the combined laser beam to move along optical path 5 forming a raster pattern at the field of view 4. A photodetector 182 which is responsive to the 740 nm frequency is provided and receives 740 nm light reflected off objects in the field of view. The photodetector 182 outputs an analog signal representing the amount of 740 nm light received. An illustrative example of a photodetector is Roithner Lasertechnik's model number EPD-740-1.

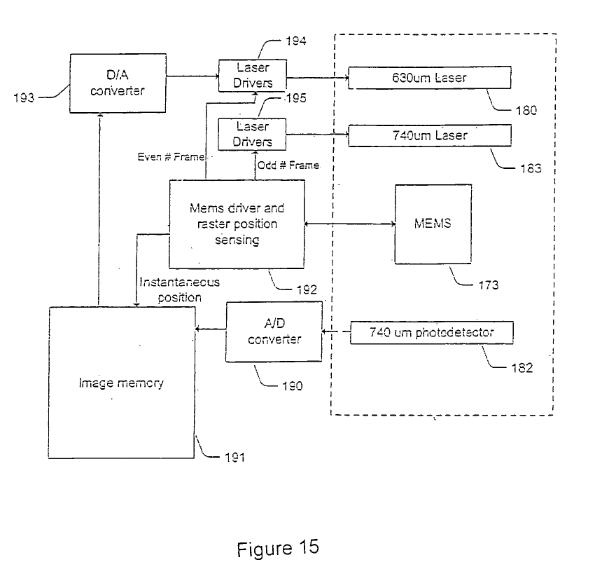

FIG. 15 shows a control block diagram for controlling the elements in FIG. 14. A first mode of operation which will be referred to hereinafter as an “Alternating Frame Mode” (AFM) follows.

In the AFM mode, an electronic block 192 for driving the MEMS driver and for sensing the position of the raster scanner is provided 192. This block generates the signals required to drive the MEMS scanner 173 in a raster pattern, and also determines the exact instantaneous location of the MEMS scanner and communicates this information to an image memory 191. This electronic block 192 also generates output signals and indicates whether the current frame (a frame is a complete raster of the field of view) is an odd number Frame 1 or an even number Frame 2 (essentially the two signals are opposite and switch polarity every other frame). The operation is as follows. The MEMS 173 is driven in a raster pattern. The first full frame after achieving a stable raster pattern will be identified as an odd number frame and the laser driver 195 for the 740 nm laser 183 is turned on for the entire frame. During this time the laser drive 194 for the 630 nm laser is turned off. The light from the 740 nm is absorbed by the veins in a patient's body and reflected by the skin of the patient, thus forming a contrasted image that is then sensed and converted into an analog signal by 740 nm photodetector 182. The analog signal is then passed through an A/D converter 190 which outputs a digital representation to image memory 191. Image memory 191 also receives instantaneous position information from the electronic block 192, and based upon such information, the digital representation is stored in a memory location corresponding to a particular pixel. This is repeated for each pixel within the odd frame. Upon completion of the odd frame, the image memory contains the image of the veins within the field of view of the MPH. During the even number frame, the laser driver 195 to the 740 nm laser is turned off. The data in the image memory 191 is read out as a function of the instantaneous position information provide by the electronic block 192 and provide to a D/A converter 193 which outputs an analog signal to laser drive 194 which drives the 630 nm laser. In this manner, the image that was stored in the odd number frame is projected by the 630 nm laser 180 in the even number frame. In this manner, the veins that are in the field of view become visible to the practitioner.

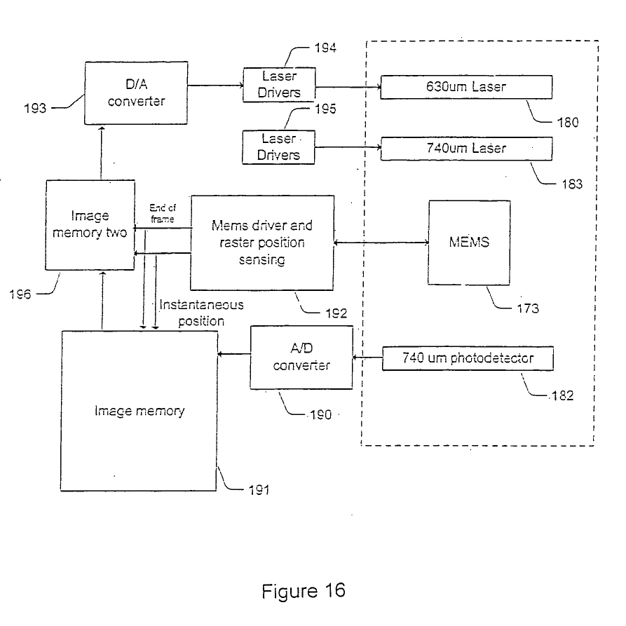

A second mode of operation is shown in FIG. 16. This mode shall be referred to hereinafter as the “Dual Buffer Mode” (DBM). In the DBM, a second image memory called image memory two 196 is added. In the DBM, the laser driver to the 740 nm laser is turned on for every frame and in each frame the image of the veins is captured and stored in image memory 191 exactly as described previously in the AFM mode. However, in this case the electronic block 192 provides an end of frame indication to both image memory two 196 and image memory 191 which causes the entire image stored in the image memory 191 to be transferred to image memory two 196 during the blanking time of the raster scan (the time after the raster scan is completed but before the next raster scan starts). During the next frame, the contents of image memory two 196 is then projected by the 630 nm laser onto the field of view. In this manner, the visible image projected is always on frame behind the actual image captured. Provided the frame rate is fast enough, this delay should not be apparent to the practitioner. Frame rates in excess of 30 frames per second can easily be achieved with the MEMS scanner provided herein.

The DBM mode is advantaged as compared to the AFM in that the visible laser is on every frame, and therefore is twice as bright. However, the AFM mode is advantaged in that it only requires a single memory buffer and therefore is more cost effective than the DBM mode.

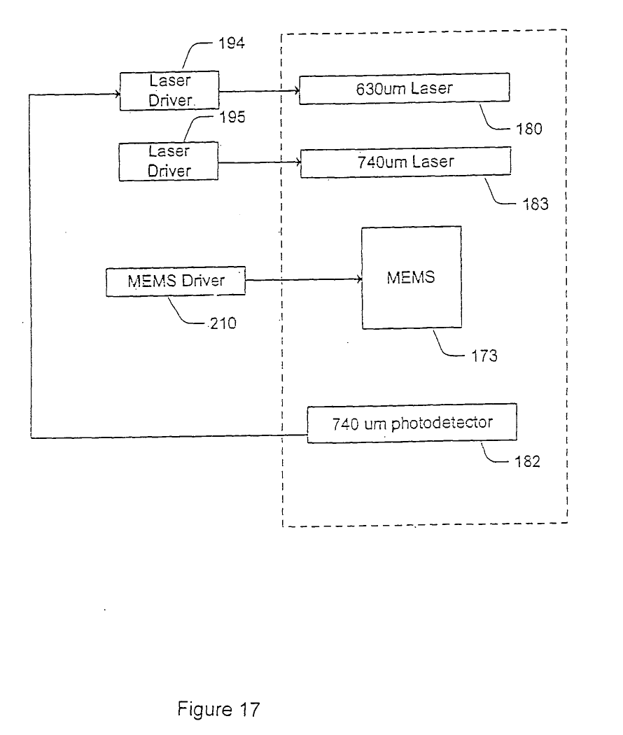

A third mode of operation is illustrated in FIG. 17. This mode shall be referred to hereinafter as the “Real Time Mode” (RTM). In the RTM the MEMS 173 is driven in a raster pattern by a MEMS driver 210. The laser driver 195 to the 740 nm laser is turned on all the time. The reflected light is received by the 740 nm photodetector 182 and the analog signal produced is connected to the laser driver 194 for the 630 nm laser 180. In this manner the red laser 180 projects nearly instantaneously the signal that is being received by the photodetector 182. The only delay is dictated by the speed of the photodetector and the laser drive 194 circuitry. Accordingly, there is no need for an image memory buffer and associated D/A converters and A/D converters. Further, since the image is never stored, there is no requirement to sense the instantaneous position of the laser for the purpose of clocking the image into memory or for projecting the visible image. In fact, in this RTM, the raster pattern does not need to be as steady and repeatable as that of the other modes, thereby possibly decreasing the complexity and cost of the MEMS and associated drive circuitry.

The RTM is so forgiving of the scan pattern that practically any dense scanning pattern can be utilized, for example, a two dimensional moving mirror is provided by Fraunhofer IPMS. In a press release dated Jan. 3, 2005 they described a two dimensional mirror as follows:

“Projection devices based on laser scanning are a very interesting alternative to matrix displays. A modulated laser and a deflection unit are necessary. Using micro scanning mirrors for implementing the laser beam deflection in a projection device has many advantages. In particular, micro scanning mirrors, which operate resonantly in both directions, enable the development of systems with very small size, high deflection angles with low voltage and low power consumption. The presented demonstrator uses a commercial laser module and a 2D micro scanning mirror operated with deflection frequencies of 9.4 kHz and 1.4 kHz. The device uses both axes to perform a sinusoidal oscillation, which causes a beam trajectory that describes a Lissajous pattern with high density, instead of the usually implemented linear scanning. Therefore, mirrors with low ratio of horizontal and vertical deflection frequency can be used. This kind of micro scanning mirrors can be fabricated easily and cost effective. The control circuit is developed with an FPGA and provides a resolution of 256.times.256 monochromatic pixels. Programmable counters are used for generating the mirror driving signals and for determining the beam position. Mirror excitation and image synchronization work without feedback loop. This means, no complicated optical or electronic synchronization techniques are needed. This simplifies micro scanning mirror and control circuit and enables low cost production. Applications of the projection device are displays, laser marking and laser exposure.”

In the RTM of FIG. 17, the MEMS could be replaced by the two-dimensional mirror of Fraunhofer IPMS which creates a Lissajous pattern with high density instead of the raster pattern. The visible laser will simply follow nearly instantaneously the image detected by the 740 nm laser detector.

In the embodiments herein the visible light transmitted was a red laser. However, any visible color or combination of color could be transmitted. For example, three laser RGB could be utilized to transmit full color images onto the field of view.

While in the embodiments herein a single two-dimensional mirror which moves in two axis was used for steering the beam, other beam steering arrangements could be used. For example, the outgoing laser beams can be bounced first off a one dimensional high speed scanning mirror and then off a second lower speed mirror scanning in the opposite direction. There are many other methods known to those skilled in the art for creating raster and other scanned laser patterns.

We claim:

1. A vein-image-enhancing system comprising: a vein scanner for creating an image of a vein of a patient; a platform for storing said scanner; and a mounting means for mounting said scanner to said platform, said mounting means permitting said scanner to be moveable from a stored position in said platform to a position being proximate to a patient.

2. The vein-image-enhancing system according to claim 1, wherein said scanner comprises: a first source of laser light and a second source of laser light, said first source of laser light transmitting laser light at a wavelength different from the wavelength transmitted by said second source of laser light; and wherein said light from said first and second sources of laser light are transmitted along a single path and directed along said path to a target; and wherein a portion of said laser light from said second source of said laser light is absorbed by said target and another portion of said laser light from said second source of laser light is reflected from said target thereby forming a contrasted image and wherein said first beam of laser light is transmitted onto the target and thereby forming a visible image representative of the contrasted image on said target.

3. The vein-image-enhancing system according to claim 2, wherein said platform comprises a hospital bed.

4. The vein-image-enhancing system according to claim 2, wherein said platform comprises an IV stand.

5. The vein-image-enhancing system according to claim 2, wherein said platform comprises a stretcher.

6. The vein-image-enhancing system according to claim 2, wherein said platform comprises a gurney.

7. The vein-image-enhancing system according to claim 2, wherein said platform comprises a wheel chair.

8. The vein-image-enhancing system according to claim 2, wherein said platform comprises an ICU boom.

9. The vein-image-enhancing system according to claim 2, wherein said platform comprises a lighting boom.

10. The vein-image-enhancing system according to claim 2, wherein said platform comprises a medical cart.

11. The vein-image-enhancing system according to claim 2, wherein said mounting means for mounting said scanner to said platform comprises a support arm, said support arm extending from a portion of said platform.

12. The vein-image-enhancing system according to claim 11, wherein said support arm is rigid.

13. The vein-image-enhancing system according to claim 11, wherein said support arm is flexible, said flexible support arm permitting deflection of said support arm to relocate said scanner to be in close proximity to a target field of view.

14. The vein-image-enhancing system according to claim 12 or 13, wherein at least a portion of said support arm is hollow to provide a conduit for passage of wiring to deliver electrical power to said device.

15. The vein-image-enhancing system according to claim 11, wherein said support arm terminates in a rigid sleeve, said rigid sleeve comprising an annular ring; and wherein said sleeve of said support arm is received in a collar in said platform, said collar comprising an annular groove, said sleeve of said support arm being received within said collar until said annular ring of said sleeve contacts said annular groove of said collar.

16. The vein-image-enhancing system according to claim 15, wherein said sleeve of said support arm is secured to said collar using a keyed lock.

17. The vein-image-enhancing system according to claim 3, wherein one end of said support arm terminates in a means of securing said support arm to one or more of: a headboard of said bed; a footboard of said bed; a side rail of said bed; and a frame of said bed.

18. The vein-image-enhancing system according to claim 13, wherein said flexible support arm may be flexed so as to locate said scanner within a storage compartment of said bed.

19. The vein-image-enhancing system according to claim 17, wherein said bed includes a connection to a source of electrical power; and wherein said storage compartment comprises electrical wiring extending from said connection to power said scanner.

20. The vein-image-enhancing system according to claim 11, wherein one end of said support arm terminates in a means of securing said support arm to one or more of: a pole of said stretcher; a handle of said stretcher; and a frame of said stretcher.

21. The vein-image-enhancing system according to claim 7, wherein one end of said support arm terminates in a means of securing said support arm to a frame of said wheel chair.

22. The vein-image-enhancing system according to claim 21, wherein said wheel chair comprises a battery pack secured to said frame, said battery pack providing power for said vein scanner.

23. The vein-image-enhancing system according to claim 8, wherein one end of said support arm terminates in a means of securing said support arm to said boom, said means being a clamp on the end of said support arm to clamp said support arm to a frame of said boom.

24. The vein-image-enhancing system according to claim 8, wherein one end of said support arm terminates in a means of securing said support arm to said boom, said means being a threaded member on the end of said support arm, said threaded member being threadably received in a threaded orifice in a portion of said boom.

25. The vein-image-enhancing system according to claim 2, wherein said laser light transmitted along said single path is coaxial.

26. The vein-image-enhancing system according to claim 25 further comprising a means for steering said laser light, said means for steering being positioned to receive said single path of laser light and move said single path of laser light to form a scanned pattern on said target.

27. The vein-image-enhancing system according to claim 34 wherein said means for steering comprises one or more mirrors.

28. The vein-image-enhancing system according to claim 27 wherein said mirror is a single biaxial mirror.

29. The vein-image-enhancing system according to claim 27 wherein there is a first mirror and a second mirror.

30. The vein-image-enhancing system according to claim 29 wherein said first and second mirrors are orthogonal to each other.

31. The vein-image-enhancing system according to claim 25 wherein at least one photo detector receives light from said second laser reflected from said target area.

32. The vein-image-enhancing system according to claim 31 wherein said photo detector outputs a detection signal which is representative of an image of said target area.

33. The vein-image-enhancing system according to claim 32 wherein there is a means for controlling said first laser as a function of said detection signal.

34. The vein-image-enhancing system according to claim 32 wherein the detection signal is stored in an image memory and wherein said image memory drives said first source of laser light.

35. The vein-image-enhancing system according to claim 32 wherein there is a first image memory and wherein said detection signal is stored in said first image memory, and wherein there is a second image memory.

36. The vein-image-enhancing system according to claim 35 wherein as said first image memory fills, the contents of said first image memory is transferred over to said second image memory and said first image memory is refilled, wherein the contents of said second image memory drives said first source of laser light.

37. The vein-image-enhancing system according to claim 31 wherein there are first and second photo detectors.

38. The vein-image-enhancing system according to claim 37 wherein said photo detectors are spatially separated.

39. The vein-image-enhancing system according to claim 2 where said first source of laser light transmits laser light in the visible spectrum.

40. The vein-image-enhancing system according to claim 39 wherein the second source of laser light transmits light in the infrared spectrum.

41. The vein-image-enhancing system according to claim 2 further comprising at least one photodetector, and a means for steering said laser light.

42. The vein-image-enhancing system according to claim 41 wherein said first and second sources of laser light, said photodetector, and said means for steering are housed in a miniature projection head.
